(19) United States
(12) Patent Application Publication
(10) Pub. No.: US 2016/0349771 A1
(43) Pub. Date: Dec. 1, 2016

(54) TEMPERATURE CONTROL APPARATUS AND METHOD IN MICROWAVE IMAGING SYSTEM

(71) Applicant: ELECTRONICS AND TELECOMMUNICATIONS RESEARCH INSTITUTE, Daejeon (KR)

(72) Inventors: Jang Yeol KIM, Daejeon (KR); Hyuk Je KIM, Daejeon (KR); Seong Ho SON, Daejeon (KR); Kwang Jae LEE, Daejeon (KR); Jong Moon LEE, Seoul (KR); Soon Ik JEON, Daejeon (KR)

(73) Assignee: ELECTRONICS AND TELECOMMUNICATIONS RESEARCH INSTITUTE, Daejeon (KR)

(21) Appl. No.: 15/079,146

(22) Filed: Mar. 24, 2016

(30) Foreign Application Priority Data
May 26, 2015 (KR) 10-2015-0072735

(51) Int. Cl. G05D 23/19; A61B 5/05; A61B 5/00; G01N 22/00

(52) U.S. Cl. CPC G05D 23/19; G01N 22/00; A61B 5/0507; A61B 5/4312; A61B 5/708

(57) ABSTRACT

Disclosed is an apparatus and a method for efficiently controlling a temperature, which are capable of minimizing a temperature deviation and decreasing a temperature control time by simultaneously controlling temperatures of matching material used in a microwave imaging system in a main water tank and an auxiliary water tank, and preventing an amplitude and phase information of the microwaves obtained by the microwave imaging system from deteriorating over a lapse of time.

### CROSS-REFERENCE TO RELATED APPLICATIONS

This application claims priority to and the benefit of Korean Patent Application No. 10-2015-0072735 filed in the Korean Intellectual Property Office on May 26, 2015, the entire contents of which are incorporated herein by reference.

### TECHNICAL FIELD

The present invention relates to an apparatus and a method of controlling a temperature of a matching material used in an imaging system using microwaves, and particularly, to an apparatus and a method of efficiently controlling a temperature for maintaining a characteristic of a matching material related to a propagation loss and a dielectric constant characteristic in a microwave imaging system.

### BACKGROUND ART

Recently, an imaging technology using microwaves has been researched by considering a sharp increase of breast cancer patients and urgent demands for new technology for a breast cancer diagnosis apparatus in a medical world, and reflecting potential growth possibility and market demands. Accordingly, the microwave imaging system using the technology is a system for diagnosing cancer by restoring an image of the breast from scattered data obtained by radiating microwaves to a breast immersed in a matching material. Accordingly, in order for an amplitude a and phase information of a signal which are the scattered data, to have accurate and stable values, a characteristic of the matching material needs to be maintained for a measurement time of signal information.

In the matching material, a propagation loss is generated during a process of radiating microwaves, and the propagation loss is changed according to a temperature. Further, a dielectric constant characteristic of the matching material is differently exhibited according to a temperature. Since a value of signal data is changed when a temperature of the matching material is changed during a process of obtaining scattered data of the breast, it is impossible to obtain a stable value, and as a result, there is a problem in restoring an image of the breast or it is impossible to restore an image of the breast. Accordingly, in order to obtain stable scattered data, an apparatus and a method of controlling a temperature for controlling a characteristic of a matching material in a microwave imaging system have been required.

### SUMMARY OF THE INVENTION

The present invention has been made in an effort to provide an apparatus and a method for efficiently controlling a temperature, which are capable of minimizing a temperature deviation and decreasing a temperature control time by simultaneously controlling temperatures of matching material used in a microwave imaging system in a main tank and an auxiliary tank, and preventing an amplitude and phase information of the microwaves, which are obtained by the microwave imaging system, from deteriorating over a lapse of time.

Technical objects of the present invention are not limited to the aforementioned technical objects and other technical objects which are not mentioned will be apparently appreciated by those skilled in the art from the following description.

An exemplary embodiment of the present invention provides an apparatus for maintaining a temperature of a water tank in a microwave imaging system, the apparatus including: a main water tank configured to contain a matching material for putting a measurement target object in the matching material; an auxiliary water tank coupled with the main water tank by a connection hose, and communicable with the matching material of the main water tank by a pump installed at a center of the connection hose; and an external computer (ex, PC) configured to perform a combined control of a first temperature control apparatus for controlling a first temperature maintaining device provided within the main water tank and a second temperature control apparatus for controlling a second temperature maintaining device provided within the auxiliary water tank(or manual control in each of the temperature control apparatus), in which the external computer obtains a microwave image by using a difference between homo microwave measurement data for the matching material of the main water tank and target microwave measurement data for the measurement target object after the measurement target object is put into the main water tank, the homo microwave measurement data and the target microwave measurement data being received from a controller for controlling sequential transception of the microwaves of a plurality of microwave transceivers installed within the main water tank by using an image restoring program.

A temperature of the matching material of each of the main water tank and the auxiliary water tank according to a control of operations of a heater and a temperature sensor included in each of the first temperature maintaining device and the second temperature maintaining device may be maintained at a predetermined temperature through the combined control of the external computer or each temperature control apparatus.

A water level sensor control device receiving the combined control of the external computer may maintain a water level of the main water tank through the communication of the matching material through the control of the pump according to sensing a water level by a water level sensor of the main water tank.

Each of the first temperature maintaining device and the second temperature maintaining device may further include a water-jet pump for circulating the corresponding matching material.

The apparatus may minimize a temperature deviation according to the communication and decrease a time taken for a temperature control, and make an amplitude and phase information of a microwave signal of the plurality of microwave transceivers be stably received by simultaneously controlling the temperatures of the matched material in the main water tank and the auxiliary water tank in the combined control of the external computer.

The measurement target object may include a phantom for a test measurement, a part of a body including a breast, or other flexible objects.

In order to obtain the homo microwave measurement data containing an amplitude and phase information of a scattered microwave signal for the matching material, before the measurement target object is put into the matching material of the main water tank, the first temperature control apparatus and the second temperature control apparatus may control the first temperature maintaining device and the second temperature maintaining device, respectively, and perform a temperature measurement, temperature setting, and a temperature control to a predetermined temperature or higher for each matching material.

In order to obtain the target microwave measurement data containing an amplitude and phase information of a scattered microwave signal for the measurement target object, in a state where the measurement target object is put into the matching material of the main water tank, the first temperature control apparatus and the second temperature control apparatus may control the first temperature maintaining device and the second temperature maintaining device, respectively, while maintaining the water level of the main water tank through the communication of the matching material through a control of the pump according to sensing a water level by a water level sensor of the main water tank under the combined control of the external computer, and perform a temperature measurement and a temperature control to a predetermined temperature or higher for the matched material, respectively.

Each of the first temperature control apparatus and the second temperature control apparatus may use any one of a proportional control, an integral control, and a differential control for controlling a temperature of the matching material thereof.

Another exemplary embodiment of the present invention provides a method of maintaining a temperature of a water tank in a microwave imaging system, the method including: uniformly maintaining a temperature of a matching material of each of a main water tank and an auxiliary water tank by using the main water tank for containing a matching material for putting a measurement target object in the matching material, the auxiliary water tank coupled with the main water tank by a connection hose, and communicable with the matching material of the main water tank by a pump installed at a center of the connection hose, and an external computer performing a combined control of a first temperature control apparatus for controlling a first temperature maintaining device provided within the main water tank and a second temperature control apparatus for controlling a second temperature maintaining device provided within the auxiliary water tank; receiving, by the external computer performing the combined control, homo microwave measurement data for the matching material of the main water tank from a controller for controlling sequential transception of the microwaves of a plurality of microwave transceivers installed within the main water tank; receiving, by the external computer performing the combined control, target microwave measurement data for the measurement target object from the controller after the measurement target object is put into the main water tank; and obtaining, by the external computer performing the combined control, a microwave image by using a difference between the homo microwave measurement data and the target microwave measurement data.

According to the apparatus and the method of controlling a temperature in the microwave imaging system according to the present invention, it is possible to minimize a temperature deviation and decrease a time taken for controlling a temperature by simultaneously controlling temperatures of matching material used in the microwave imaging system in the main water tank and the auxiliary water tank, prevent an amplitude and phase information of microwaves received by the microwave imaging system from deteriorating over a lapse of time, and easily restore a microwave image of an object, such as the breast, by stably and accurately obtaining scattered data.

The present invention may be widely utilized in a breast cancer diagnosis apparatus using a matching material, a cancer diagnosis in a medical device field, a converged system of a biomedical field and a radio communication field, other diagnosis device fields utilizing body tissue, and the like.

### BRIEF DESCRIPTION OF THE DRAWINGS

FIG. 1 is a configuration diagram of a microwave imaging system including a temperature control apparatus according to an exemplary embodiment of the present invention.

FIG. 2 is a diagram illustrating an example of an amplitude and phase information of received microwaves according to a change in a temperature in the microwave imaging system according to the exemplary embodiment of the present invention.

FIG. 3 is a flowchart for describing a method of restoring an image for a measurement target object according to the exemplary embodiment of the present invention.

FIG. 4 is a flowchart for describing a method of controlling a temperature of a matching material according to an exemplary embodiment of the present invention.

It should be understood that the appended drawings are not necessarily to scale, presenting a somewhat simplified representation of various features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter, some exemplary embodiments of the present invention will be described in detail with reference to the accompanying drawings. When reference numerals refer to elements of each drawing, it is noted that although the same elements are illustrated in different drawings, the same elements are referred to by the same reference numerals as much as possible. Further, in describing the exemplary embodiments of the present invention, when it is determined that the detailed description of publicly known configurations or functions related to the present invention may obscure the understanding of the exemplary embodiment of the present invention, the detailed description thereof will be omitted.

In describing constituent elements of the exemplary embodiment of the present invention, terms such as first, second, A, B, (a), (b), and the like may be used. Such a term is only for discriminating the constituent element from another constituent element, and does not limit the essential feature, order, or sequence of the constituent element, or the like. Further, otherwise contrarily defined, all terms used herein including technological or scientific terms have the same meaning as those generally understood by those skilled in the art. Terms which are defined in a generally used dictionary should be interpreted to have the same meaning as the meaning in the context of the related art but are not interpreted as ideal or excessively formal meaning otherwise clearly defined in the present invention.

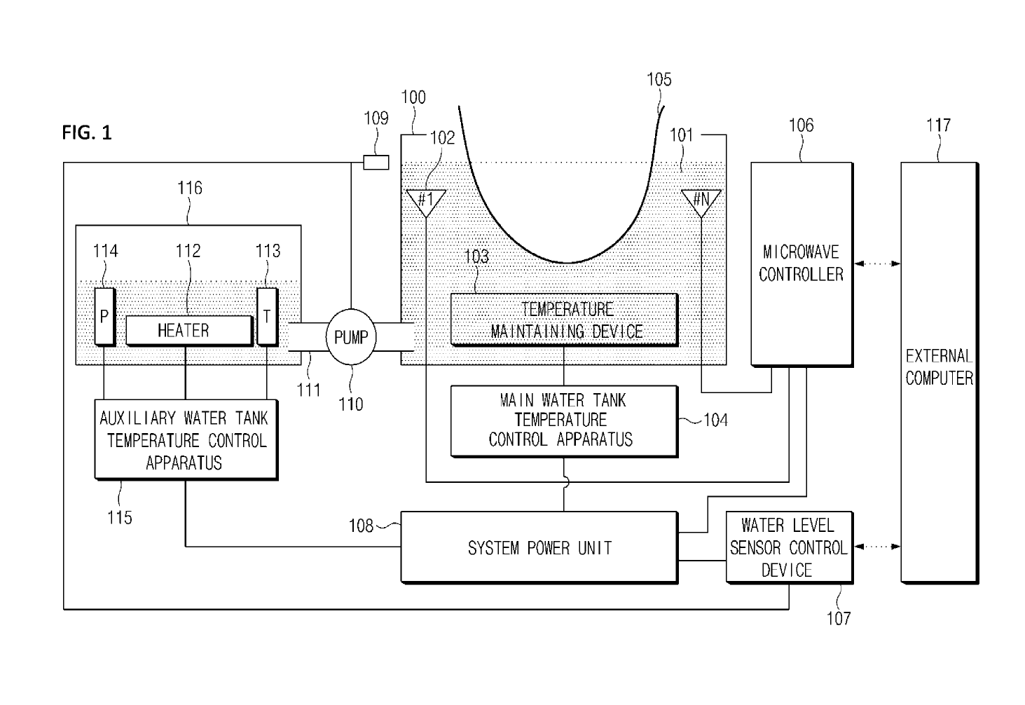

FIG. 1 is a configuration diagram of a microwave imaging system including a temperature control apparatus according to an exemplary embodiment of the present invention.

Referring to FIG. 1, the microwave imaging system according to the exemplary embodiment of the present invention includes a main water tank 100 and an auxiliary water tank 116 for putting a matching material 101, and the main water tank 100 includes a temperature maintaining device 103 and N microwave transceivers 102 (N is a natural number) therein, and the auxiliary water tank 116 also includes a temperature maintaining device (including a heater 112, a temperature sensor 113, a water-jet (or circulation) pump 114, and the like) therein. The main water tank 100 and the auxiliary water tank 116 are connected by a connection hose 111, and a pump 110 for supplying and discharging the matching material 101 is provided at a center of the connection hose 111.

The microwave imaging system according to the exemplary embodiment of the present invention includes a main water tank temperature control apparatus 104 controlling the temperature maintaining device 103 of the main water tank 100, and an auxiliary water tank temperature control apparatus 115 controlling the temperature maintaining devices 112, 113, and 114 of the auxiliary water tank 116. The main water tank temperature control apparatus 104 and the auxiliary water tank temperature control apparatus 115 may be manually controlled by control devices thereof, respectively, and are controlled under a combined control of an external computer (for example, a personal computer) 117 and receives power from a system power unit 108. A microwave transception controller 106 may transmit microwaves to the N microwave transceivers 102 or receive microwaves from the N microwave transceivers 102 under the combined control of the external computer 117. The combined control of the external computer 117 may control a general operation of the microwave imaging system. In addition, the combined control of the external computer 117 may control a water level sensor control device 107, and the water level sensor control device 107 may be connected with the pump 110 and a water level sensor 109 and transceive a control signal necessary for the operations. The matching material 101 communicates (the material is movable from one side to the other side) between the main water tank 100 and the auxiliary water tank 116 according to an operation of the pump 110, and the external computer 117 may control the matching material 101 to be moved by operating the pump 110 so that a water level of the matching material 101 of the main water tank 100 is maintained at a predetermined height through the water level sensor control device 107 in the combined control of the external computer 117 according to a detection signal of the water level sensor 109.

The N microwave transceivers 102 may have a form including a microwave radiating unit having a structure of a waveguide form and a predetermined aperture for inputting/outputting electromagnetic waves, such as microwaves. A measurement target object 105 is put in the matching material 101, and the N microwave transceivers 102 may be disposed around the measurement target object 105 at an appropriate interval. The matching material 101 may be a predetermined liquid through which microwaves pass through well, and the measurement target object 105 may be a flexible object, such as a phantom for a test measurement or a part of a body, such as a breast, or other objects, of which images are measurable.

In order to obtain a microwave image for the measurement target object 105, under the combined control (a combined control program, which is software, is available) of the external computer 117, the microwave transception controller 106 controls the N microwave transceivers 102 to sequentially transmit microwaves once from any one predetermined microwave transceiver, and collects information about the microwaves, which the remaining transceivers, other than the corresponding microwave transceiver transmitting the microwaves, simultaneously receive. The information (microwave measurement data) about the microwaves received by the transceivers contains an amplitude and phase information of a microwave signal, which passes through the measurement target object 105 and is scattered, and the N microwave transceivers 102 may sequentially transmit microwaves once, and the information about the microwaves received by the remaining transceivers may be collected by the microwave transception controller 106. An image restoring program (software) of the external computer 117 may analyze the information about the microwaves collected by the microwave transception controller 106, obtain a microwave image for the measurement target object 105, and utilize the obtained microwave image for diagnosing a cancer and the like.

In this case, a propagation loss of the microwave passing through the measurement target object 105 may be generated in a corresponding frequency region by a characteristic of the matching material 101, and the propagation loss is generated by a characteristic of a unique dielectric constant and conductivity of the matching material 101 and the characteristics of the dielectric constant and the conductivity are changed by a change in a temperature of the matching material 101, so that a propagation loss characteristic may be varied. Accordingly, a signal analysis of the received microwave may be inaccurate due to a change in a temperature, and there is difficulty in restoring a microwave image of the measurement target object 105.

Accordingly, in the present invention, it is possible to minimize a temperature deviation and decrease a time taken for controlling a temperature by simultaneously controlling temperatures of the matching material 101 in the main water tank 100 and the auxiliary water tank 116, prevent an amplitude and phase information of the received microwave from deteriorating over a lapse of time, and easily restore a microwave image of the measurement target object 105, such as the breast, by stably and accurately obtaining scattered data.

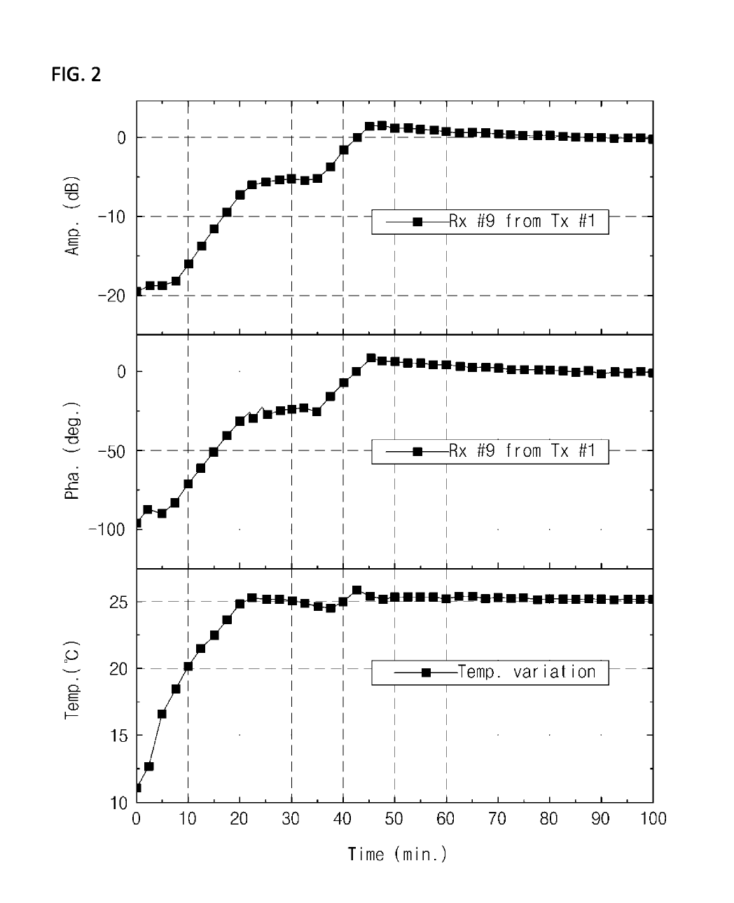

FIG. 2 is a diagram illustrating an example of an amplitude and phase information of microwaves received by the N microwave transceivers 102 according to a change in a temperature in the microwave imaging system according to the exemplary embodiment of the present invention. Here, 16 microwave transceivers 102 are used, which includes a first transmission channel Tx#1 and the remaining 15 reception channels including a reception channel Rx#9, which is farthest from the first transmission channel Tx#1, and it is assumed that a temperature of the matching material 101 is 11.6 to 25° C., and a time is 100 minutes.

As illustrated in FIG. 2, it can be seen that when a temperature (Temp.) of the matching material 101 is changed over a lapse of time (around 40 mins), an amplitude (Amp.) and phase (Pha.) information of the received microwaves are irregularly changed, so that it is difficult to accurately generate a microwave image. Accordingly, in the present invention, it is possible to uniformly maintain a temperature (Temp.) of the matching material 101 over a lapse of time (after around 40 mins) according to the operations of the main water tank temperature control apparatus 104, the temperature maintaining device 103, the auxiliary water tank temperature control apparatus 115, and the temperature maintaining devices 112, 113, and 114, and thus it is possible to stably and accurately obtain an amplitude (Amp.) and phase (Pha.) information about the received microwave by uniformly maintaining a dielectric constant and conductivity of the matching material 101 without degrading the amplitude (Amp.) and the phase (Pha.) information about the received microwave, thereby generating a reliable microwave image for the measurement target object 105.

For example, during the measurement of the measurement target object 105, the auxiliary water tank temperature control apparatus 115 may heat a solution of the matching material 101 within the auxiliary water tank 116 by supplying power of the system power unit 108 to the heater 112, such as a ring type sheath heater operable in water, by using a thermostat and the like, and generating heat, and maintain a temperature of the matching material 101 at a predetermined temperature by controlling a supply of the power to the heater 112 to be on/off according to a sensed temperature (or signal) obtained through the temperature sensor 113 (the number of temperature sensors 113 may be two or more, and a height of a connection part of an end of the sensor is variable so that a height of the end of the sensor is adjustable according to a water level) installed within the auxiliary water tank 116. Further, the auxiliary water tank temperature control apparatus 115 may control the matching material 101 to generally and evenly have a predetermined temperature by making the matching material 101 within the auxiliary water tank 116 be forcibly circulated by controlling an operation of the water-jet pump 114 during the measurement.

Similarly, although not illustrated in the drawing, the temperature maintaining device 103 of the main water tank 100 may include a heater, a temperature sensor (the number of temperature sensors may be two or more, and a height of a connection part of an end of the sensor is variable so that a height of the end of the sensor is adjustable according to a water level), a water-jet pump, and the like having the aforementioned forms. Accordingly, the main water tank temperature control apparatus 104 may heat the solution of the matching material 101 of the main water tank 100 by supplying the power of the system power unit 108 to the heater of the temperature maintaining device 103 and generating heat, and control the supply of the power to the corresponding heater to be on/off according to the sensed temperature (or signal) obtained through the temperature sensor (the number of temperature sensors may be two or more) of the temperature maintaining device 103 installed in the main water tank 100 to maintain the matching material 101 at a predetermined temperature. Further, the main water tank temperature control apparatus 104 may control the matching material 101 to generally and evenly have a predetermined temperature by making the matching material 101 within the main water tank 100 be forcibly circulated by controlling the operation of the water-jet pump of the temperature maintaining device 103 during the measurement.

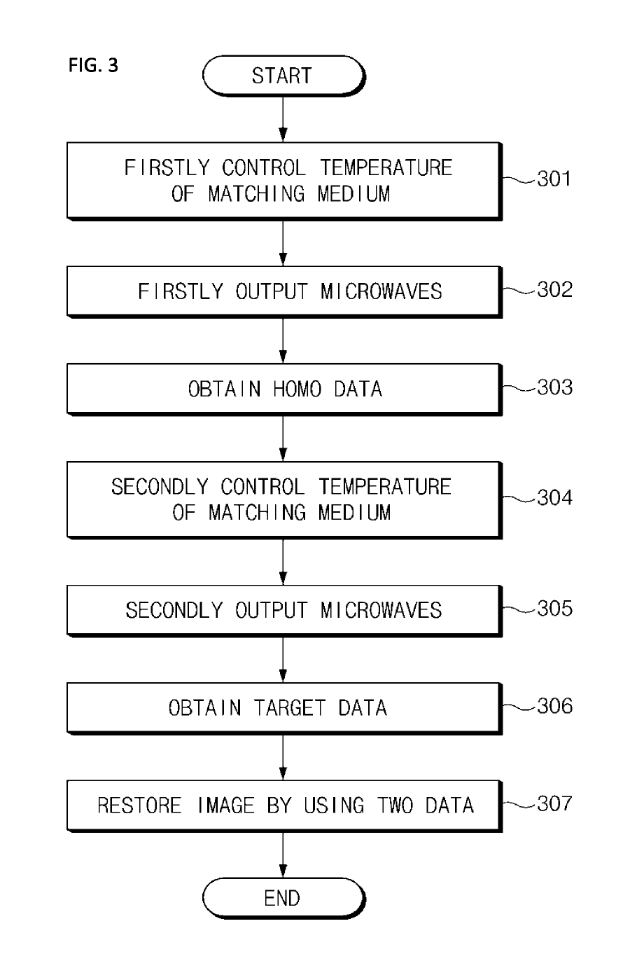

FIG. 3 is a flowchart for describing a method of restoring an image for the measurement target object 105 according to the exemplary embodiment of the present invention.

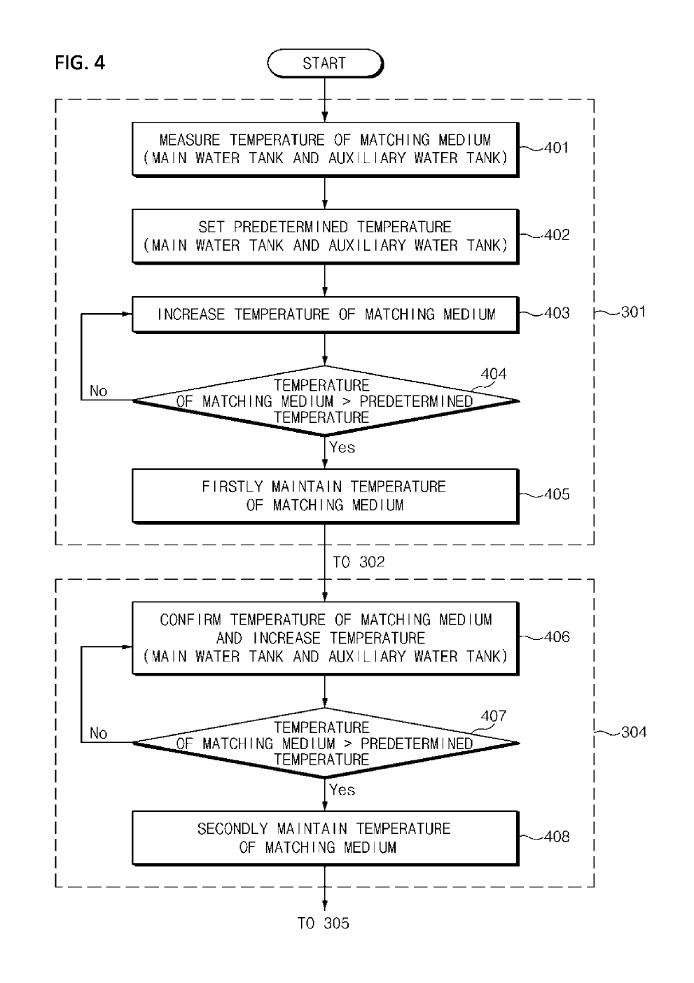

In order to obtain a microwave image for the measurement target object 105, first, in a state where the measurement target object 105 is not put in the matching material 101, the main water tank temperature control apparatus 104 controls the temperature maintaining device 103 so that the matching material 101 of the main water tank 100 is maintained at a predetermined temperature (301). A method of controlling a temperature of the matching material 101 (first control of a temperature of the matching material) will be described in more detail with reference to FIG. 4 (401 to 405).

When the matching material 101 is maintained at the predetermined temperature in the state where the measurement target object 105 is not put in the matching material 101 as described above, the microwave transception controller 106 controls the N microwave transceivers 102 to sequentially transmit microwaves once from any one predetermined microwave transceiver (first microwave output) under the combined control of the external computer 117 (302), and collects information (an amplitude and phase information about a microwave signal, which passes only the matching material and is scattered) about the microwaves, which the remaining transceivers, other than the corresponding microwave transceiver transmitting the microwaves, simultaneously receive (303). The information about the received microwaves in this case corresponds to homo data (microwave measurement data) for the matching material 101 in the state where the measurement target object 105 is not put in the matching material 101.

When the aforementioned homo data is obtained, the matching material 101 of the main water tank 100 is moved to the auxiliary water tank 116 by controlling an operation of the pump 110 through the water level sensor control device 107 in the combined control of the external computer 117 according to a sensed signal of the water level sensor 109 before or after the measurement target object 105 is put in the matching material 101, so that a water level of the main water tank 100 is controlled to be maintained at a predetermined height (see the description of operation 406 of FIG. 4). The main water tank temperature control apparatus 104 controls the temperature maintaining device 103 so that the matching material 101 of the main water tank 100 is maintained at the predetermined corresponding temperature with the adjustment of the water level at the same time or after the adjustment of the water level, and the auxiliary water tank temperature control apparatus 115 controls the temperature maintaining devices 112, 113, and 114 so that the matching material 101 of the auxiliary water tank 116 is maintained at the predetermined corresponding temperature (the same temperature as that of the matching material 101 of the main water tank 100) (304). The method of controlling a temperature of the matching material 101 (second control of a temperature of the matching material) will be described in more detail with reference to FIG. 4 (operations 406 to 408).

When the matching material 101 is maintained at the predetermined temperature in the state where the measurement target object 105 is put in the matching material 101 as described above, the microwave transception controller 106 controls the N microwave transceivers 102 to sequentially transmit microwaves once from any one predetermined microwave transceiver (second microwave output) under the combined control of the external computer 117 (305), and collects information (an amplitude and phase information about a microwave signal, which passes the measurement target object 105 and is scattered) about the microwaves, which the remaining transceivers, other than the corresponding microwave transceiver transmitting the microwaves, simultaneously receive (306). The information about the received microwaves in this case corresponds to target data (microwave measurement data) for the matching material 101 in the state where the measurement target object 105 is put in the matching material 101.

The image restoring program of the external computer 117 may analyze information, that is, the homo data and the target data, about the microwaves collected by the microwave transception controller 106, and obtain a microwave image for the measurement target object 105 (307). That is, the image restoring program of the external computer 117 may generate a microwave image for the measurement target object 105 by calculating a difference (subtraction) value between the target data and the homo data.

FIG. 4 is a flowchart for describing a method of controlling a temperature of a matching material according to an exemplary embodiment of the present invention. Operations 401 to 405 of FIG. 4 may be included in operation 301 of FIG. 3. Further, operations 406 to 408 of FIG. 4 may be included in operation 304 of FIG. 3, and in this case, in the setting of a predetermined temperature for controlling a temperature of the matching material 101, setting temperatures of the main water tank 100 and the auxiliary water tank 116 are equally set.

First, in a state where the measurement target object 105 is not put in the matching material 101, water levels of the matching material 101 in the main water tank 100 and the auxiliary water tank 116 are not changed and are set under an optimum condition. In this case, it is possible to measure temperatures of an upper part and a lower part of the main water tank 100 by using the temperature sensor of the temperature maintaining device 103 of the main water tank 100 under the control of the main water tank temperature control apparatus 104 (401). Particularly, in the measurement of the temperature, it is possible to measure a temperature of an upper part of the matching material 101 adjacent to the plurality of transceivers 102 and a temperature of a lower part of the matching material 101 adjacent to the heater positioned at a lower part of the main water tank 100. Further, it is possible to measure a temperature of the matching material 101 around the heater 112 of the auxiliary water tank 116 by using the temperature sensor 113 under the control of the auxiliary water tank temperature control apparatus 115. Further, the temperatures measured from the main water tank 100 and the auxiliary water tank 116 may be displayed on the temperature control apparatuses 104 and 115, respectively, and may also be displayed in real time on a combined control window of a display device of the external computer 117 depending on a case.

In order to obtain accurate signal data based on the temperature of the matching material 101 measured as described above, a user may set setting temperatures of the main water tank 100 and the auxiliary water tank 116 by using the temperature control apparatuses 104 and 115, respectively, or the combined control window of the external computer 117 (402). Further, the user may set an operation of the water-jet pump of the temperature maintaining device 103 of the main water tank 100 by using the temperature control apparatus 104 or the combined control window of the external computer 117. Particularly, a temperature deviation of the matching material 101 at the upper part and the lower part of the main water tank 100 exists, and in order to minimize the temperature deviation during the temperature control process, the user may set the operation of the water-jet pump. Further, in the setting of the operation of the water-jet pump 114 of the auxiliary water tank 116, the water-jet pump 114 may be operated by setting a predetermined operation setting value according to a water level of the matching material 101 put in the auxiliary water tank 116 in the auxiliary water tank temperature control apparatus 115 or on the combined control window of the external computer 117. That is, when a water level of the matching material 101 of the auxiliary water tank 116 is high, the user may input a setting value so that the water-jet pump 114 is operable for circulating the matching material 101 and maintaining the predetermined temperature, and when a water level of the matching material 101 of the auxiliary water tank 116 is low, the user may input a setting value so that the water-jet pump 114 is not operated. Further, at least one control method among proportional integral differential temperature control methods including a proportional control, an integral control, and a differential control for controlling the temperatures of the matched material 101 of the main water tank 100 and the auxiliary water tank 116 may be set in the respective temperature control apparatuses 104 and 115 or the combined control window of the external computer 117.

After the temperature is set as described above, it is possible to increase the temperatures of the matched material 101 of the main water tank 100 and the auxiliary water tank 116 up to the predetermined corresponding temperatures by operating the heaters within the respective water tanks under the control of the respective temperature control apparatuses 104 and 115 through the combined control of the external computer 117 (403).

The temperature control apparatuses 104 and 115 increase the temperatures of the matched material 101 by operating the heaters until the temperatures of the matched material 101 become the predetermined corresponding temperatures while confirming whether the temperatures of the matched material 101 of the main water tank 100 and the auxiliary water tank 116 are equal to or higher than the predetermined corresponding temperatures based on the sensing of the temperature by the temperature sensors thereof through the combined control of the external computer 117 (404).

Through the repetition of the aforementioned process, the temperatures of the matched material 101 of the main water tank 100 and the auxiliary water tank 116 may be maintained at the predetermined corresponding temperatures or higher (405). The aforementioned process may be performed by operating each water-jet pump by the respective temperature control apparatuses 104 and 15 or under the combined control of the external computer 117.

In this case, since the matching material 101 of the auxiliary water tank 116 does not need the adjustment of a water level with the matching material 101 of the main water tank 100, so that a process of adjusting a water level for supplying or discharging the matching material 101 is not required. Accordingly, in a situation where the matching material 101 of the main water tank 100 is maintained at the predetermined temperature, regardless of the situation where the matching material 101 of the auxiliary water tank 116 is maintained at the predetermined temperature, the microwave imaging system performs operation 302 of FIG. 3 of outputting microwaves by using the plurality of transceivers 102 in order to obtain the homo data containing the amplitude and the phase information of the signal for the matching material 101 in the situation where the measurement target object 105 is not present in the main water tank 100.

In the meantime, after the homo data is obtained in operations 302 and 303 of FIG. 3, in order to obtain the target data for the measurement target object 105, the matching material temperature control, which is operation 304 of FIG. 3, is performed again. Further, operations 406 to 408 of FIG. 4 may be included in operation 304 of FIG. 3, and in this case, in the setting of a predetermined temperature for controlling a temperature of the matching material 101, setting temperatures of the main water tank 100 and the auxiliary water tank 116 are equally set.

In order to put the measurement target object 105 in the matching material 101 of the main water tank 100, an optimized water level of the matching material 101 positioned at a higher water level sensor 109 of the main water tank 100 needs to be decreased. Accordingly, a process of discharging the matching material 101 to the auxiliary water tank 116 by using a discharge pump 110 and the connection hose 111 is performed up to a point, at which the water level sensor 109 is positioned. The matching material 101 of the main water tank 100 may be discharged to the auxiliary water tank 116 partially or up to a predetermined position of the water level sensor 109 before the measurement target object 105 is put in the matching material 101, or the matching material 101 may also be supplied to the main water tank 100 up to a predetermined position of the water level sensor 109 after the measurement target object 105 is put in the matching material 101. The appropriate predetermined positions of the water level sensor 109 before and after the measurement target object 105 is put in the matching material 101 may be manually controlled or be controlled under the combined control of the external computer 117. In order to meet the water level of the matching material 101 up to the position of the water level sensor 109 through the control of the pump 110 by the water level sensor control device 107 under the combined control of the external computer 117, the matching material 101 may be moved to the auxiliary water tank 116, as well as the main water tank 100, and the temperatures of the matching material 101 for both the main water tank 100 and the auxiliary water tank 116 are simultaneously controlled as described above, so that it is possible to minimize a temperature deviation according to the communication of the matching material 101 between the main water tank 100 and the auxiliary water tank 116 and decrease a time taken for the temperature control.

When the measurement target object 105 is put in the main water tank 100, and it is confirmed that the matched material of the main water tank 100 and the auxiliary water tank 116 are mixed by performing a process of supplying the matching material 101 of the auxiliary water tank 116 to the main water tank 100 up to the position of the water level sensor 109 of the main water tank 100 by using the pump 110 and the connection hose 11, each temperature control apparatus 104 or 105 measures a temperature of each water tank, and the main water tank temperature control apparatus 104 controls the temperature maintaining device 103 so that the matching material 101 of the main water tank 100 is maintained at a predetermined corresponding temperature, and the auxiliary water tank temperature control apparatus 115 controls the temperature maintaining devices 112, 113, and 114 so that the matching material 101 of the auxiliary water tank 116 is maintained at a predetermined corresponding temperature (the same temperature as that of the matching material 101 of the main water tank 100) (406). After the matched material are mixed, it is possible to increase the temperatures of the matched material 101 of the main water tank 100 and the auxiliary water tank 116 up to the predetermined corresponding temperatures by operating the heaters within the respective water tanks under the control of the respective temperature control apparatuses 104 and 115.

The temperature control apparatuses 104 and 115 increase the temperatures of the matched material 101 by operating the heaters until the temperatures of the matched material 101 become the predetermined corresponding temperatures while confirming whether the temperatures of the matched material 101 of the main water tank 100 and the auxiliary water tank 116 are equal to or higher than the corresponding predetermined temperatures based on the sensing of the temperature by the temperature sensors thereof (407). Through the repetition of the aforementioned process, the temperatures of the matched material 101 of the main water tank 100 and the auxiliary water tank 116 may be maintained at the predetermined corresponding temperatures or higher (408). The aforementioned process may be performed by operating the water-jet pumps by the temperature control apparatuses 104 and 115, respectively.

When the temperatures are maintained as described above, the microwave imaging system performs operation 305 of FIG. 3 of outputting microwaves by using the plurality of transceivers 102 in order to obtain the target data containing the amplitude and phase information of the signal for the matching material 101 in the situation where the measurement target object 105 is present in the main water tank 100.

According to the microwave imaging system including the temperature control apparatus according to the present invention, it is possible to minimize a temperature deviation and decrease a time taken for controlling a temperature by simultaneously controlling temperatures of matching material in the main water tank and the auxiliary water tank, prevent an amplitude and phase information of microwaves received by the microwave imaging system from deteriorating over a lapse of time, and easily restore a microwave image of an object, such as the breast, by stably and accurately obtaining scattered data. The present invention may be widely utilized in a breast cancer diagnosis apparatus using a matching material, a cancer diagnosis in a medical device field, a converged system of a biomedical field and a radio communication field, other diagnosis device fields utilizing body tissue, and the like.

The foregoing has been described for purposes of illustrating the technical spirit of the present invention, and those skilled in the art may change and modify the present invention in various ways without departing from the essential characteristic of the present invention.

Accordingly, the various exemplary embodiments disclosed herein are not intended to limit the technical spirit but describe with the true scope and spirit being indicated by the following claims. The scope of the present invention should be construed based on the following appended claims and it should be construed that the technical spirit included within the scope equivalent to the claims belongs to the present invention.

What is claimed is:

1. An apparatus for maintaining a temperature of a water tank in a microwave imaging system, the apparatus comprising:
a main water tank configured to contain a matching material for putting a measurement target object in the matching material;
an auxiliary water tank coupled with the main water tank by a connection hose, and communicable with the matching material of the main water tank by a pump installed at a center of the connection hose; and
an external computer configured to perform a combined control of a first temperature control apparatus for controlling a first temperature maintaining device provided within the main water tank and a second temperature control apparatus for controlling a second temperature maintaining device provided within the auxiliary water tank,
wherein the external computer performing the combined control obtains a microwave image by using a difference between homo microwave measurement data for the matching material of the main water tank and target microwave measurement data for the measurement target object after the measurement target object is put into the main water tank, the homo microwave measurement data and the target microwave measurement data being received from a controller for controlling sequential transception of the microwaves of a plurality of microwave transceivers installed within the main water tank.

2. The apparatus of claim 1, wherein the external computer performing the combined control maintains a water level of the main water tank through the communication of the matching material through the control of the pump according to sensing a water level by a water level sensor of the main water tank through a water level sensor control device, and maintains a temperature of the matching material of each of the main water tank and the auxiliary water tank according to a control of an operation of a heater and a temperature sensor included in each of the first temperature maintaining device and the second temperature maintaining device at a predetermined temperature.

3. The apparatus of claim 2, wherein each of the first temperature maintaining device and the second temperature maintaining device further includes a water-jet pump for circulating the corresponding matching material.

4. The apparatus of claim 1, wherein the external computer performing the combined control minimizes a temperature deviation according to the communication and decreases a time taken for a temperature control, and makes an amplitude and phase information of a microwave signal of the plurality of microwave transceivers be stably received by simultaneously controlling the temperatures of the matched material in the main water tank and the auxiliary water tank.

5. The apparatus of claim 1, wherein the measurement target object includes a phantom for a test measurement, a part of a body including a breast, or other flexible objects.

6. The apparatus of claim 1, wherein in order to obtain the homo microwave measurement data containing an amplitude and phase information of a scattered microwave signal for the matching material, before the measurement target object is put into the matching material of the main water tank, the first temperature control apparatus and the second temperature control apparatus control the first temperature maintaining device and the second temperature maintaining device, respectively, and perform a temperature measurement, temperature setting, and a temperature control to a predetermined temperature or higher for each matching material.

7. The apparatus of claim 1, wherein in order to obtain the target microwave measurement data containing an amplitude and phase information of a scattered microwave signal for the measurement target object, in a state where the measurement target object is put into the matching material of the main water tank, the first temperature control apparatus and the second temperature control apparatus control the first temperature maintaining device and the second temperature maintaining device, respectively, while maintaining the water level of the main water tank through the communication of the matching material through a control of the pump according to sensing a water level by a water level sensor of the main water tank through a water level sensor control device under the combined control of the external computer, and perform a temperature measurement and a temperature control to a predetermined temperature or higher for the matched material, respectively.

8. The apparatus of claim 1, wherein each of the first temperature control apparatus and the second temperature control apparatus uses one or more of a proportional control, an integral control, and a differential control for controlling a temperature of the matching material thereof.

9. A method of maintaining a temperature of a water tank in a microwave imaging system, the method comprising:
uniformly maintaining a temperature of a matching material of each of a main water tank and an auxiliary water tank by using the main water tank for containing a matching material for putting a measurement target object in the matching material, the auxiliary water tank coupled with the main water tank by a connection hose, and communicable with the matching material of the main water tank by a pump installed at a center of the connection hose, and an external computer performing a combined control of a first temperature control apparatus for controlling a first temperature maintaining device provided within the main water tank and a second temperature control apparatus for controlling a second temperature maintaining device provided within the auxiliary water tank;
receiving, by the external computer performing the combined control, homo microwave measurement data for the matching material of the main water tank from a controller for controlling sequential transception of the microwaves of a plurality of microwave transceivers installed within the main water tank;
receiving, by the external computer performing the combined control, target microwave measurement data for the measurement target object from the controller after the measurement target object is put into the main water tank; and
obtaining, by the external computer performing the combined control, a microwave image by using a difference between the homo microwave measurement data and the target microwave measurement data.

10. The method of claim 9, wherein the combined control of the external computer includes maintaining a water level of the main water tank through the communication of the matching material through the control of the pump according to sensing a water level by a water level sensor of the main water tank through a water level sensor control device, and maintaining a temperature of the matching material of each of the main water tank and the auxiliary water tank according to a control of an operation of a heater and a temperature sensor included in each of the first temperature maintaining device and the second temperature maintaining device at a predetermined temperature.

11. The method of claim 10, wherein each of the first temperature maintaining device and the second temperature maintaining device further includes a water-jet pump for circulating the corresponding matching material.

12. The method of claim 9, wherein the external computer performing the combined control minimizes a temperature deviation according to the communication and decreases a time taken for a temperature control by simultaneously controlling the temperatures of the matched material in the main water tank and the auxiliary water tank.

13. The method of claim 9, wherein the measurement target object includes a phantom for a test measurement, a part of a body including a breast, or other flexible objects.

14. The method of claim 9, wherein in order to obtain the homo microwave measurement data containing an amplitude and phase information of a scattered microwave signal for the matching material, before the measurement target object is put into the matching material of the main water tank, the first temperature control apparatus and the second temperature control apparatus control the first temperature maintaining device and the second temperature maintaining device, respectively, and perform a temperature measurement, temperature setting, and a temperature control to a predetermined temperature or higher for each matching material.

15. The method of claim 9, wherein in order to obtain the target microwave measurement data containing an amplitude and phase information of a scattered microwave signal for the measurement target object, in a state where the measurement target object is put into the matching material of the main water tank, the first temperature control apparatus and the second temperature control apparatus control the first temperature maintaining device and the second temperature maintaining device, respectively, while maintaining the water level of the main water tank through the communication of the matching material through a control of the pump according to sensing a water level by a water level sensor of the main water tank through a water level sensor control device under the combined control of the external computer, and perform a temperature measurement and a temperature control to a predetermined temperature or higher for the matched material, respectively.

16. The method of claim 9, wherein each of the first temperature control apparatus and the second temperature control apparatus uses one or more of a proportional control, an integral control, and a differential control for controlling a temperature of the matching material thereof.
